(19) United States
(12) Patent Application Publication
(10) Pub. No.: US 2026/0314938 A1
(43) Pub. Date: Oct. 15, 2026

(54) DEVICE FOR MEASURING THE TEMPERATURE OF A HUMAN BODY PART

(71) Applicant: Markus Geffe, Duisburg (DE)

(72) Inventor: Markus Geffe, Duisburg (DE)

(21) Appl. No.: 19/482,607

(22) PCT Filed: Apr. 22, 2024
(86) PCT No.: PCT/DE2024/200030

§ 371 Date: Nov. 7, 2025

(30) Foreign Application Priority Data
May 10, 2023 (DE) 10 2023 204 316.5

(51) Int. Cl. A61B 5/01; G01J 5/00

(52) U.S. Cl. CPC A61B 5/01; G01J 5/0025; A61B 2562/0271

(57) ABSTRACT

A device for measuring the temperature of a human body part, in particular a hand and/or a foot, having a receptacle for the body part and a sensing apparatus, including at least one temperature sensor, for determining the temperature, in particular the surface temperature, of at least one region of the body part situated on the receptacle, is provided, wherein a cooling device that uses a coolant to cool the body part is situated on and/or at the receptacle.

### BACKGROUND

### Technical Field

The present disclosure relates to a device for measuring the temperature of a human body part, in particular a hand and/or a foot.

### Description of the Related Art

In the field of medical research, the capability for drawing conclusions on existing illnesses, based on temperature measurements of a human body surface, is already known. For example, in so-called thermography the surface temperature of a region of the body is depicted, and assessed by a medical practitioner with regard to the presence of a pathological change.

In recent years, it has been found, for example, that rheumatoid arthritis may be detected even in the early stage by sensing the physiological warming of a hand over a period of time after stimulation by cold. For this purpose, the hand is placed on a table, beneath a thermal imaging camera. The thermal imaging camera is fixed on a stand above the hand. If rheumatoid arthritis is present, the existing inflammation in the hand results in uneven warming, which in the ideal case is deducible from the recorded temperature profile. In particular, it can be displayed that the inflamed joints heat up more quickly than the surrounding tissue.

However, it is problematic that the recordings or data obtained with the known arrangement are apparently subject to disturbance variables, so that the reliability is not high enough to allow a pathological change in the temperature profile to be identified.

### BRIEF SUMMARY AND GENERAL DESCRIPTION

Therefore, the present disclosure provides and refines a device for measuring the temperature of a human body part in such a way that a reliable and meaningful measurement may be achieved using simple design means.

Accordingly, a device for measuring the temperature of a human body part, in particular a hand and/or a foot, is described herein. The device has a receptacle for the body part and a sensing apparatus, including at least one temperature sensor, for determining the temperature, in particular the surface temperature, of at least one region of the body part situated on the receptacle. A cooling device uses a coolant to cool the body part situated on and/or at the receptacle.

According to the present disclosure, it has been found advantageous to provide a cooling device by way of which the body part situated on or at the receptacle may be stimulated by cold. This yields an advantage that after the stimulation by cold, the body part does not have to be moved, and instead remains at the receptacle so that the physiological warming is then measurable by the sensing apparatus. By use of the sensing apparatus, an extremely accurate temperature profile of the body part or a region of the body part may be recorded, in particular during the warming of the body part.

By use of the device according to the present disclosure, it is possible not only to carry out precise cooling of the body part, but also to always keep the body part in a precisely defined position, namely, at or on the receptacle. As a result of this design measure, disturbance variables which may arise due movement or inaccurate positioning of the body part and imprecise cooling are prevented. The data obtained via the sensing apparatus are therefore also particularly suited for evaluation by software with regard to the presence of an illness, preferably rheumatoid arthritis, in particular based on artificial intelligence. Regardless of the manner in which the data are evaluated, in addition to temperature data the data may involve geometric data, i.e., data concerning the geometric surface properties of the body part, in particular the hand or the foot.

At this point it is noted that the device according to the disclosure is suited in particular for detecting rheumatoid arthritis, but is not limited to this use. It is also conceivable to detect other changes or illnesses using the data obtained from the sensing apparatus, in particular temperature data and/or geometric data.

Furthermore, the body part may in particular be a hand and/or a foot. It is conceivable and advantageous for the receptacle and the sensing apparatus to be designed in such a way that determination of the temperature of the hand, including the wrist, or of the foot, including the ankle, is made possible. In addition, it is advantageous for the cooling device to be designed in such a way that cooling of the hand, including the wrist, or of the foot, including the ankle, is made possible by the coolant.

The receptacle may advantageously have at least one opening. It is thus possible for a side of the accommodated body part facing the receptacle, in particular a hand surface, to not be covered, at least in part, by the receptacle. The coolant can thus come into direct contact with a larger surface area of the body part, and in addition a larger region of the body part is available for a measurement. The receptacle may in particular be a support for the body part, for example a hand rest that is in particular ergonomically shaped.

More advantageously, the receptacle may have at least one fixing element to hold the body part in a defined position. Such a design has the advantage that, in particular during the temperature measurement, movements of the body part that represent a disturbance variable in the obtained temperature data are prevented or at least reduced. For example, a bracket, a loop, or the like may be used as a fixing element.

The coolant may particularly advantageously have a temperature in the range of −10° C. to 20° C. It is thus possible to cool the body part to the required target temperature in a relatively short time. The coolant may particularly advantageously have a temperature in the range of 0° C. to 10° C., so that on the one hand relatively rapid cooling is possible, and on the other hand the coolant is not perceived as painful by a subject. Regardless of the temperature, the coolant may be water or some other liquid, for example. Alternatively or additionally, a gaseous medium may be used as coolant.

According to one advantageous embodiment, the cooling device may have a basin which accommodates the coolant. Such an embodiment is suited in particular when a liquid coolant is used. It is conceivable for the basin to be provided separately, for example as a Dewar flask, and/or to have at least one active cooling element, for example a Peltier element. According to a further advantageous embodiment, a device for monitoring the temperature of the coolant could be provided to allow the coolant to always be held at the desired temperature. Alternatively or additionally, the basin could have an inflow and an outflow for the coolant.

More advantageously, the cooling device may have at least one outlet nozzle for leading the coolant to the receptacle and/or to the body part. Use of an outlet nozzle is particularly advantageous when a gas is utilized as coolant.

The cooling device may particularly advantageously have an enclosure element for the body part, in which a space, for example a channel, for the coolant is formed. In particular, it is conceivable for the enclosure element to be designed as a glove having a double-wall design, for example, so that the coolant is accommodated in a closed space. The space may have a multipart design, for example a two-part design, so that the glove is divided into a top side and a bottom side, through both of which the coolant may flow independently.

Furthermore, it is conceivable and advantageous when the cooling device or the basin may be placed between a neutral position in which there is little or no action by the coolant on the receptacle and/or the body part, and a cooling position in which the receptacle and/or the body part are/is acted on by the coolant. It is thus not necessary to move the body part, so that resulting disturbance variables in the temperature measurement are avoided.

According to at least one advantageous embodiment, the sensing apparatus may have multiple temperature sensors. It is thus possible to carry out the temperature measurement at various angles with respect to the receptacle and/or the body part. The temperature data obtained with such an arrangement are particularly well suited for creating a temperature profile of the examined body part. Regardless of the number of temperature sensors, it is conceivable that it/they is/are arrangeable at different positions, for example displaceably or fixably in a form-fit and/or force-fit manner at the appropriate points.

At least two temperature sensors may advantageously be arranged in such a way that a temperature measurement is achievable from a side of the body part facing away from the receptacle, for example a back of the hand, and from a side of the body part facing the receptacle, for example a hand surface. Based on the data or temperature data recorded from the back of the hand and the hand surface, for example, an illness-related, i.e., “abnormal,” warming may be detected in an improved manner so that a more reliable diagnosis may be made. There is the further advantage that the sensing apparatus and the body part are not moved during the data collection, thus avoiding errors in the data or image recording.

More advantageously, the sensing apparatus may be rotatable about the receptacle. Thus, temperature measurements may be made from different regions of the body part.

According to a further advantageous embodiment, the temperature sensor may be a thermal imaging camera. The images recorded with a thermal imaging camera are particularly well suited for evaluation with regard to the presence of an illness, in particular rheumatoid arthritis. It is also is conceivable for the temperature sensor to be a pyroelectric sensor. This has an advantage that temperatures are precisely measurable. Alternatively or additionally, the sensing apparatus may have at least one camera, in particular an RGB camera.

It is advantageously conceivable for the sensing apparatus to have at least one surface-detecting sensor, for example a lidar sensor. Such a design measure has the advantage that the surface geometry of the body part is detectable, so that for example the intensity of swelling, for example of a finger, is identifiable.

The sensing apparatus may advantageously have at least one sensing unit with a thermal imaging camera, and a camera associated with same. Arranging a “conventional camera,” for example an RGB camera, directly next to the thermal imaging camera has the advantage that the label function of the thermographic data is automatable or optimizable. In particular, a so-called automatic finger knuckle annotation of the temperature data may take place. The camera or the RGB camera makes this possible due to the fact that the image data enable identification of the “drape” of folds of the skin above the joints, for example for the dorsal view (view of the back of the hand), and of the “creases” beneath the joints for the ventral view (view of the palm). The particular folds cannot be depicted by the data that are collected by a thermal imaging camera. It is also conceivable for the thermal imaging camera and the camera associated with it to form a structural unit, i.e., to have a one-piece or combined design.

Moreover, a computing device, for example a computer, may be provided for processing the measured values ascertained by the sensing apparatus. The computing device may be used to control or operate the individual components of the device (sensing apparatus and/or movement of the cooling device, etc.). The evaluation of the measured values may take place directly in the computing device. It is also conceivable for the computing device to transfer at least a portion of the recorded data, in particular temperature data and/or geometric data, or data that it has preprocessed, to a further computing device, for example a cloud, in which all or part of the evaluation of the data, in particular temperature data and/or geometric data, takes place. The computing device may be designed to evaluate the recorded data, in particular temperature data and/or geometric data, using statistical methods. According to one advantageous embodiment, it is conceivable for the computing device to be designed to evaluate the recorded data, in particular temperature data and/or geometric data, using artificial intelligence for pattern recognition.

It is also possible for the computing device to be designed to supply the recorded data, in particular temperature data and/or geometric data, for diagnostic refinement of pattern recognition that is being learned and further developed, and to use same to evaluate the data. For evaluation of a precise prediction, a customized algorithm that assesses the data sets collected from the subjects and which claims to increase precision and character delimitation by self-learning may be validated. A conceptual model based on actual training data may be developed using common models. Geometric data sets, in conjunction with the data of the particular measurements of the body part collected by the sensing apparatus, could be interpreted and methodically evaluated in two dimensions and in context, also collectively across data sets.

More advantageously, a pull-off device may be provided for removing a protective element, for example a glove, from the body part situated on and/or at the receptacle. The protective element is used to ensure that the body part to be examined is not wetted with the coolant, for example is not moist, after the cooling, since this could interfere with the temperature measurement.

According to a further advantageous embodiment, the sensing apparatus may include at least one further sensor for determining a physiological parameter selected from electrical conductivity of the skin, oxygen saturation of the blood, pulse and heart rhythm, and concentration of erythrocytes, leukocytes, lymphocytes, and hemoglobin (Hb).

More advantageously, an analysis device may be provided, via which measuring strips for analyzing applied blood drops may be at least partially inserted. Disease-relevant inflammatory proteins, antibodies, uric acid, pathogens or further blood values may thus be determined, so that even further information concerning the patient's state of health may be collected and also the presence of rheumatoid arthritis may be further determined. Typical inflammation values, among other things, could also be determined in this way: C-reactive protein (CRP), typical antibodies, including antibodies against cyclic citrullinated peptides (anti-CCP) and rheumatoid factor (RF), as well as typical organic values such as alanine aminotransferase (ALT), aspartate aminotransferase (AST), and creatinine (crea).

### BRIEF DESCRIPTION OF THE SEVERAL VIEWS OF THE DRAWINGS

There are various options for advantageously developing and refining the teaching of the present disclosure. In this regard, reference is made to exemplary embodiments of the disclosure based on the drawings. In conjunction with the explanation of exemplary embodiments of the disclosure, based on the drawings, generally preferred embodiments and refinements of the teaching are also explained. In the figures:

FIG. 1 shows a schematic illustration of one exemplary embodiment of a device according to the disclosure,

FIGS. 2a and 2b show further schematic illustrations of the exemplary embodiment according to FIG. 1,

FIGS. 3a through 3c show further schematic illustrations of the exemplary embodiment according to FIG. 1,

FIGS. 4a and 4b show further schematic illustrations of the exemplary embodiment according to FIG. 1,

FIGS. 5a and 5b show further schematic illustrations of the exemplary embodiment according to FIG. 1,

FIGS. 6a and 6b show further schematic illustrations of the exemplary embodiment according to FIG. 1,

FIG. 7 shows an enlarged detail view of a portion of the device according to FIG. 1,

FIG. 8 shows another enlarged detail view of the portions of the device according to FIG. 1 shown in FIG. 7, and

FIG. 9 shows a schematic illustration of the arrangement of a receptacle and a sensing apparatus of a device according to the disclosure.

### DETAILED DESCRIPTION

It is noted that for the sake of improved clarity, in each figure every component is not necessarily provided with the associated reference numeral.

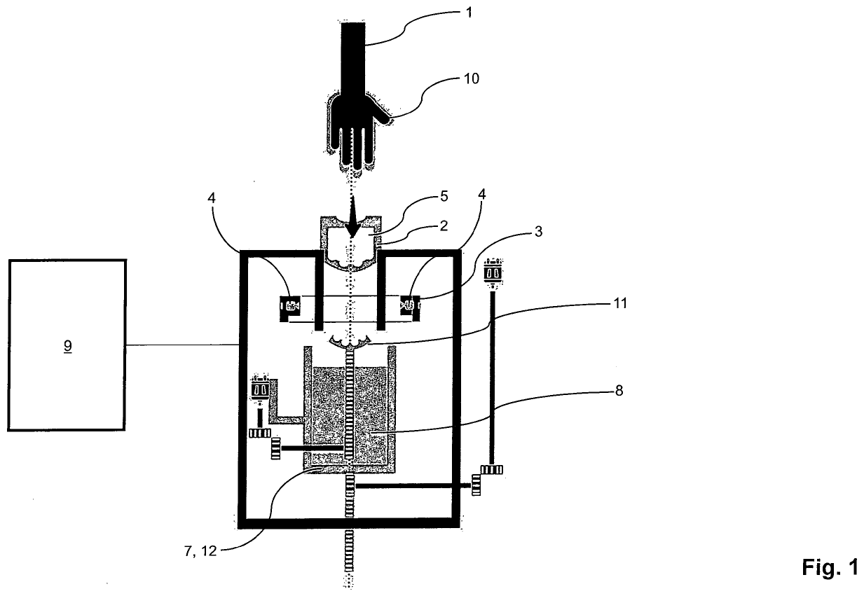

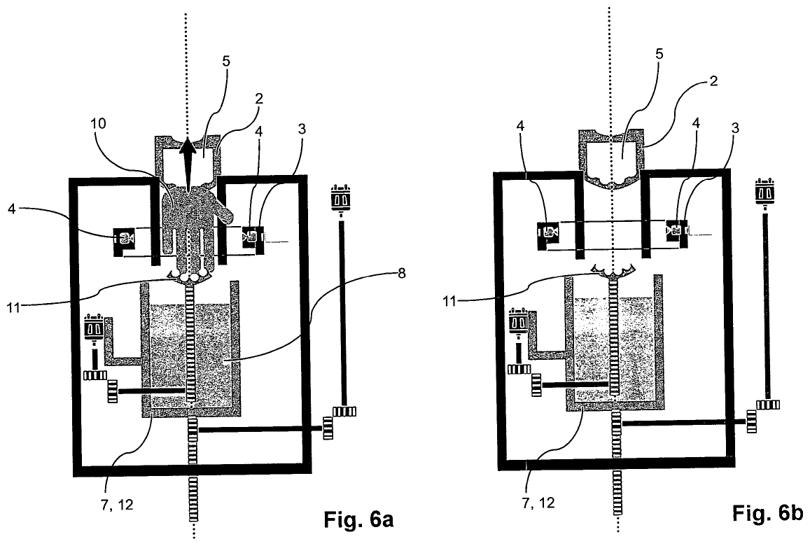

FIGS. 1 through 8 show one exemplary embodiment of a device according to the disclosure for measuring the temperature of a human body part 1. In the illustrated exemplary embodiment here, the body part 1 is a hand.

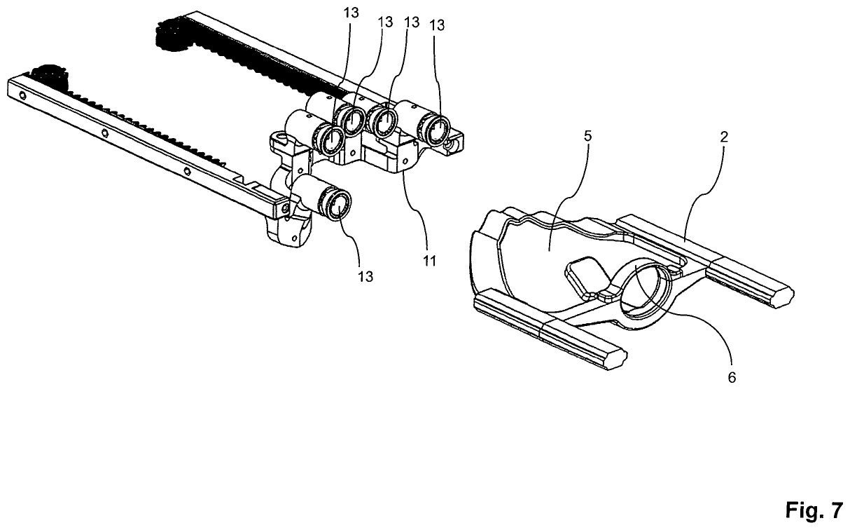

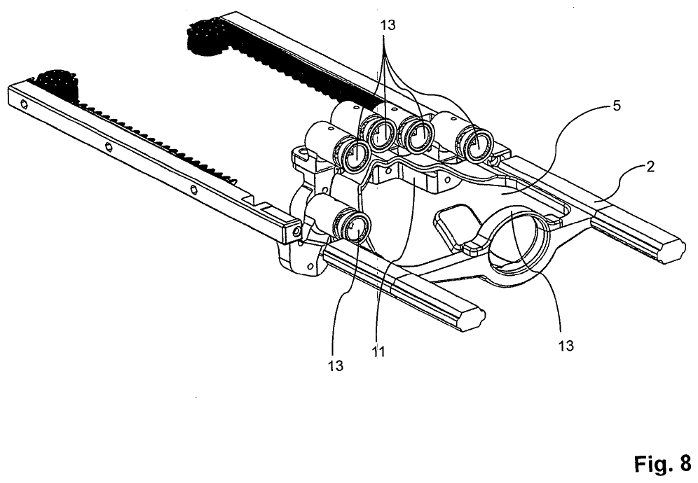

The device has a receptacle 2 for the body part 1 as well as a sensing apparatus 3 with two temperature sensors 4. More than two temperature sensors 4 or only a single temperature sensor 4 may also be provided. It is apparent in particular in FIGS. 7 and 8 that the receptacle 2 has a large opening 5, so that the placed body part 1, observed in the ventral view (view of the palm), is covered as little as possible. The temperature sensors 4 may be thermal imaging cameras, for example. In addition, a fixing element 6 is provided at the receptacle 2 in order to hold the body part 1 in the desired position during use and to prevent movement as much as possible.

Furthermore, a cooling device 7 is provided, via which the body part 1 may be acted on by a coolant 8 in order to cool the body part 1 to a defined temperature or surface temperature, for example. For this purpose the cooling device 7 includes a basin 12 which accommodates the coolant 8.

In addition, illustrated only in FIG. 1 is a computing device 9 that is used for processing or evaluation of the data, in particular temperature data and/or geometric data, obtained from the temperature sensors and any other sensors, cameras, etc., and optionally used for controlling individual elements of the device.

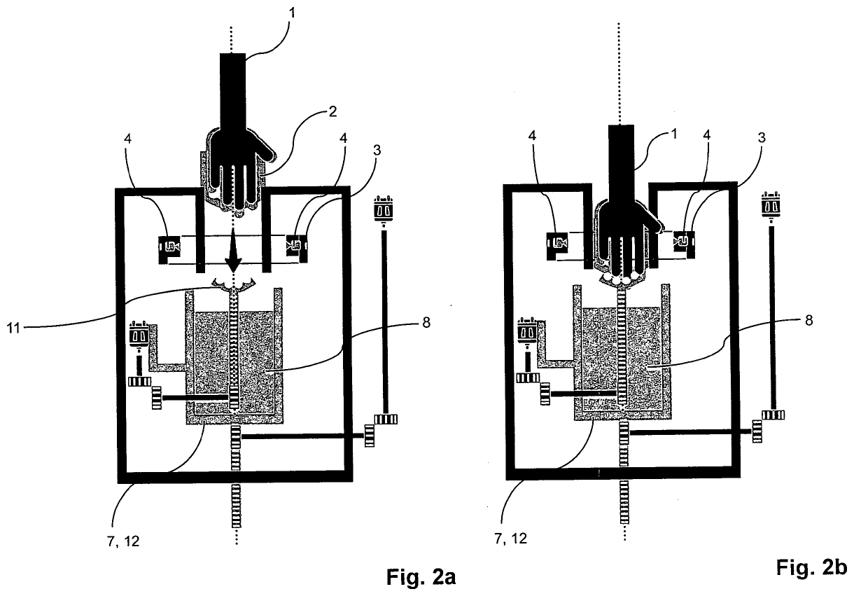

The course of a temperature measurement is apparent from FIGS. 1 through 6b. Accordingly, first the body part 1 is situated on or at the receptacle 2. The body part is enclosed by a protective element 10, in the present case a glove (see FIG. 1). It is pointed out that a protective element 10 does not necessarily have to be provided, although this is advantageous. The receptacle 2 may then be introduced into the device (see FIGS. 2a and 2b). It is also conceivable for the receptacle 2 to be immovable. For example, the receptacle could be fixed in the position illustrated in FIG. 2b.

FIGS. 1 through 8 also show a pull-off device 11 that is used to remove the protective element 10, wherein the device does not necessarily have to have a pull-off device 10 (see FIG. 2b).

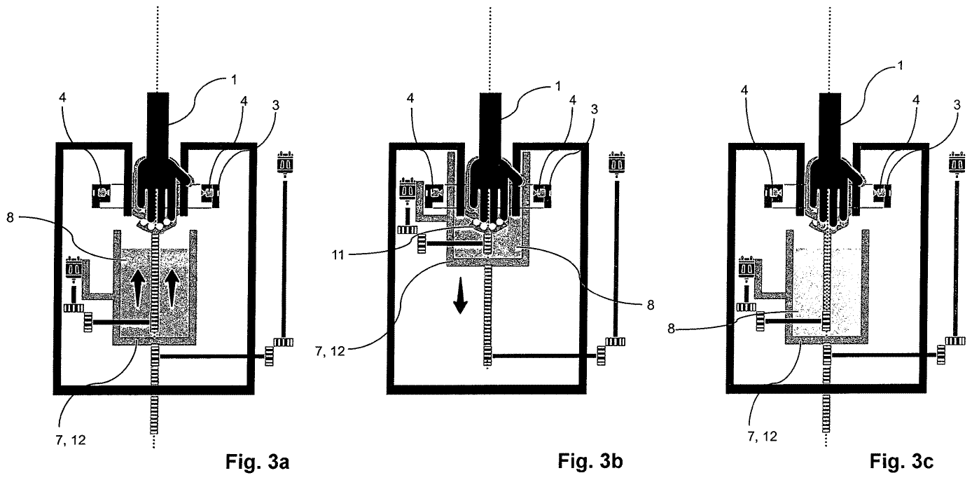

According to FIGS. 3a and 3b, the basin 12 of the cooling device 7 is brought from the neutral position into a cooling position in which the coolant 8 is in contact with the body part 1 in order to cool it to the desired temperature. The basin 12 does not necessarily have to have a movable design, and other designs are conceivable. For example, the basin 12 could be fixed in the cooling position illustrated in FIG. 3b, and could have an inflow and an outflow so that coolant 8 may flood the basin 12 and be emptied therefrom.

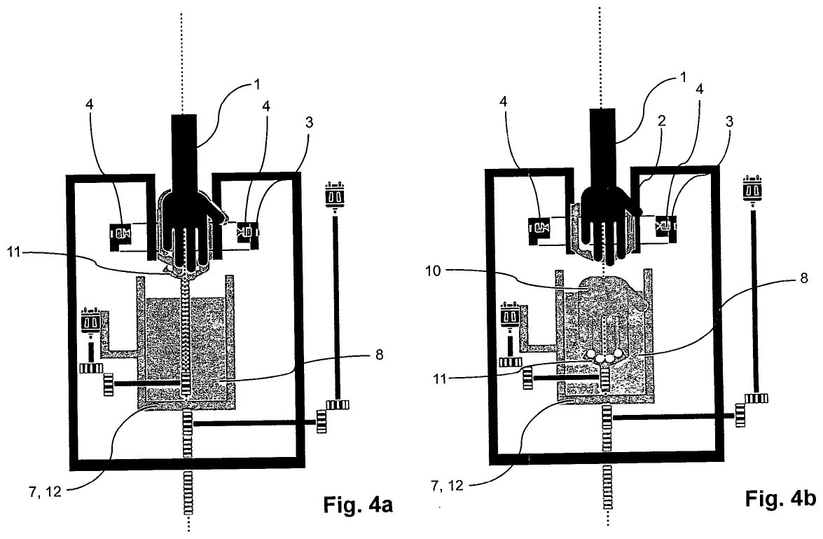

FIG. 3c shows that after the body part 1 has been cooled, the cooling device 7 is once again brought into the neutral position. It is apparent In FIGS. 4a and 4b how the protective element 10 is removed from the body part by the pull-off device 11. As described above, a protective element 10 and thus the pull-off device 11 are not absolutely necessary.

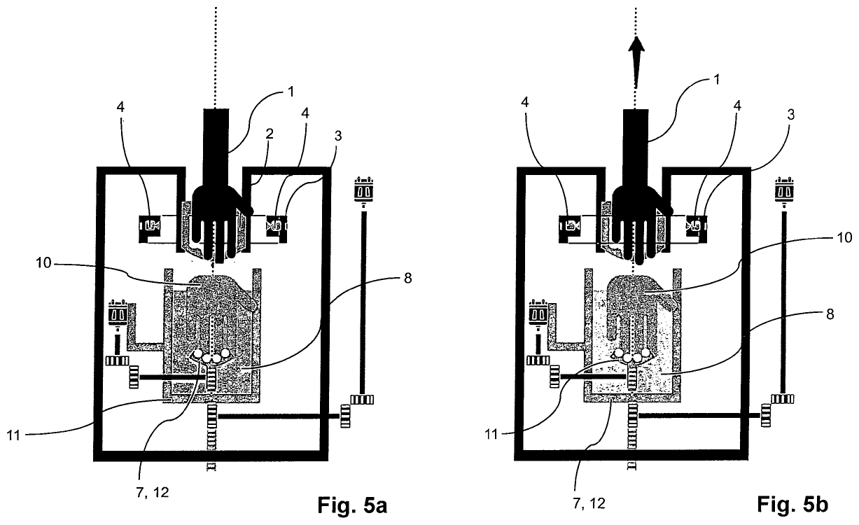

FIG. 5a shows the step in which the temperature of at least one region of the body part is detected by the temperature sensors 4 during the physiological warming of the body part 1. In other words, the surface temperature of the body part 1 is sensed over a period of time, in particular in such a way that the warming of the joints and also of the respective surrounding tissue is detected. The data thus obtained may be directly evaluated by the computing device 9 or transferred to a further computing device for evaluation. The evaluation preferably takes place using an artificial intelligence-based method. After the measurement is completed, the body part 1 is removed from the receptacle 2 (FIG. 5b). By use of the described device it is thus possible to precisely cool a body part 1 to a defined temperature in order to then record a temperature profile during the warming. It is important that the body part 1 is subject to no movement, so that based on the determined temperature profiles a statement concerning the presence of an illness, in particular rheumatoid arthritis, may be made in a particularly reliable manner.

FIGS. 7 and 8 show an enlarged detail view of the receptacle 2 and the pull-off device 11. It is clearly apparent that the receptacle 2 is ergonomically adapted to a hand surface, wherein due to the opening 5, only a small portion of the body part 1, i.e., a hand, is covered by the receptacle 2. This has the further advantage that the temperature of the receptacle has only a negligible effect on the warming of the body part 1. It is also illustrated that the pull-off device 11 has five pull-off elements 13, each of which engages with the distal end of a respective finger in order to pull off a protective element 10, namely, a glove, from the body part 1.

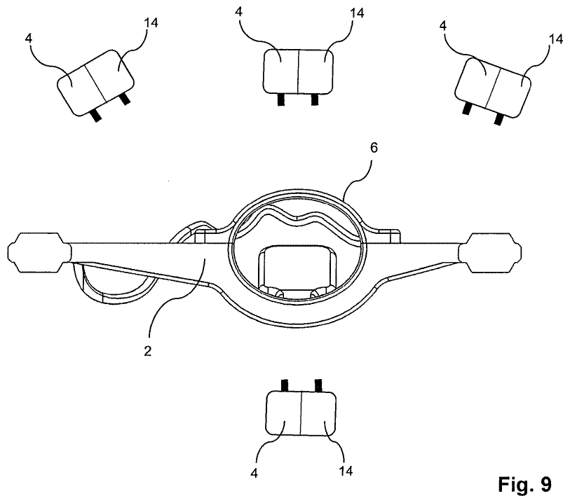

FIG. 9 shows an example of one possible arrangement of a sensing apparatus 3 with respect to a receptacle 2. Such an arrangement may be provided in the device shown in FIGS. 1 through 8. The combination of the above statements regarding FIGS. 1 through 8 and FIG. 9 thus expressly represents a part of the present disclosure.

According to FIG. 9, the temperature sensors 4, for example thermal imaging cameras, are each provided in combination with a “customary” camera 14, for example an RGB camera. These combinations of temperature sensor 4 and camera 14 are positioned with respect to the receptacle 2 in such a way that temperature measurements may be made by the three temperature sensors 4 shown at the top in FIG. 9 from a side of the body part 1 facing away from the receptacle 2, for example a back of the hand. A side of the body part 1 facing the receptacle 2, for example a hand surface, may make temperature measurements by the temperature sensor 14 shown at the bottom in FIG. 9. It is also conceivable for the temperature sensors 4, for example thermal imaging cameras, to be arranged corresponding to FIG. 9, but for no cameras 14 to be provided or for not all temperature sensors 4 to be arranged in each case in combination with a camera 14. In addition, one or more cameras 14 could be positioned around the receptacle 2, independently of the temperature sensors 4.

With regard to further advantageous embodiments of the device according to the disclosure, reference is made to the general part of the description and to the appended claims in order to avoid repetition

Lastly, it is expressly pointed out that the embodiments of the device according to the disclosure described above serve only to explain the claimed teaching, but do not restrict it to the exemplary embodiments.

### LIST OF REFERENCE NUMERALS

1 body part
2 receptacle
3 sensing apparatus
4 temperature sensor
5 opening
6 fixing element
7 cooling device
8 coolant
9 computing device
10 protective element
11 pull-off device
12 basin
13 pull-off element
14 camera

The various embodiments described above can be combined to provide further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled.

1. A device for measuring the temperature of a human body part, comprising:
a receptacle for the body part;
a sensing apparatus including at least one temperature sensor, for determining a temperature of at least one region of the body part situated on or at the receptacle; and
a cooling device that uses a coolant to cool the body part,
wherein the cooling device is situated on and/or at the receptacle.

2. The device according to claim 1, wherein the receptacle has at least one opening so that a side of the body part facing the receptacle is not covered, at least in part, by the receptacle.

3. The device according to claim 1, wherein the receptacle has at least one fixing element to hold the body part in a defined position.

4. The device according to claim 1, wherein the coolant has a temperature in a range of −10° C. to 30° C.

5. The device according to claim 1, wherein the cooling device has a basin which accommodates the coolant.

6. The device according to claim 1, wherein the cooling device can be placed between a neutral position in which there is little or no action by the coolant on the receptacle and/or the body part, and a cooling position in which the receptacle and/or the body part are/is acted on by the coolant.

7. The device according to claim 1, wherein the sensing apparatus has multiple temperature sensors so that a temperature measurement may be carried out at various angles with respect to the receptacle and/or the body part.

8. The device according to claim 1, wherein at least two temperature sensors are arranged such a that a temperature measurement is achievable from a side of the body part facing away from the receptacle, and from a side of the body part facing the receptacle.

9. The device according to claim 1, wherein the sensing apparatus is rotatable about the receptacle.

10. The device according to claim 1, wherein:
the at least one temperature sensor is a thermal imaging camera and/or a pyroelectric sensor, and/or
the sensing apparatus has at least one camera, and/or
the sensing apparatus has at least one surface-detecting sensor.

11. The device according to claim 10, wherein the sensing apparatus has at least one sensing unit with a thermal imaging camera, and a camera associated with same.

12. The device according to claim 1, further comprising a computing device configured to process measured values ascertained by the sensing apparatus.

13. The device according to claim 1, further comprising a pull-off device configured to remove a protective element from the body part situated on and/or at the receptacle.

14. The device according to claim 1, wherein the temperature is a surface temperature of the at least one region of the body part.

15. The device according to claim 4, wherein the temperature of the coolant is in a range of 0° C. to 20° C.

16. The device according to claim 1, wherein the cooling device has at least one outlet nozzle for leading the coolant to the receptacle and/or to the body part.

17. The device according to claim 1, wherein the cooling device has an enclosure element that includes a space for the coolant.

18. The device according to claim 10, wherein the at least one camera is an RBG camera.

19. The device according to claim 10, wherein the at least one surface-detecting sensor is a lidar sensor.

20. The device according to claim 13, wherein the protective element is a glove, and the pull-off device is configured to remove the glove from the body part.
